(19) United States
(12) Patent Application Publication
(10) Pub. No.: US 2021/0358242 A1
(43) Pub. Date: Nov. 18, 2021

(54) Quarantine Gate Apparatus For Supporting Quarantine Measures For A Facility To Be Accessed By Multiple Persons In An Non-Contact Manner

(71) Applicant: Weon Kook KIM, Andong-si (KR)

(72) Inventor: Weon Kook KIM, Andong-si (KR)

(21) Appl. No.: 17/017,848

(22) Filed: Sep. 11, 2020

(30) Foreign Application Priority Data
May 13, 2020 (KR) 10-2020-0056836
May 19, 2020 (KR) 10-2020-0059708
Jun. 1, 2020 (KR) 10-2020-0065595

(51) Int. Cl. G07C 9/25; G07C 9/27; A61L 2/24; A61L 2/00

(52) U.S. Cl. CPC G07C 9/257; G07C 9/27; A61L 2/24; A61L 2202/15; G07C 2209/08; A61L 2202/14; A61L 2/0094

(57) ABSTRACT

A quarantine gate apparatus to be disposed at a boundary of a facility to be accessed by multiple users is provided. The quarantine gate apparatus includes an inner part bordering on inside of the facility, an outer part bordering on outside of the facility and a conditional opening gate disposed between the inner part and the outer part. An entering person can reach the inside of the facility by sequentially passing through the outer part, the conditional opening gate, and the inner part. The quarantine gate apparatus includes a body temperature obtainer, a body temperature analyzer, an identification information receiver, an identification information storage, and a gate controller.

### CROSS-REFERENCE TO RELATED APPLICATIONS

This application claims priority to Korean Patent Application No. 10-2020-0056836 filed on May 13, 2020, Korean Patent Application No. 10-2020-0059708 filed on May 19, 2020, Korean Patent Application No. 10-2020-0065595 filed on Jun. 1, 2020, the entire contents of which are herein incorporated by reference.

### BACKGROUND

### 1. Field

Exemplary embodiments of the present disclosure relate to a quarantine management gate apparatus disposed at an access boundary of a facility that may be accessed by multiple persons (e.g. various multi-use facilities, such as public facilities or commercial facilities). More particularly, exemplary embodiments of the present disclosure relate to a quarantine management gate apparatus disposed at the access boundary of the facility, which may provide (dry and wet) sterilization/disinfection treatment for each person passing therethrough to enter the facility and may conveniently collect identification information thereon in an non-contact digital manner so that the collected identification information may be suitably used in a timely manner for a special public benefit such as public health and quarantine.

### 2. Description of the Related Art

Recently, pandemic diseases are occurring more and more frequently. In particular, pandemic diseases caused by new viruses or germs, for which no preventive or therapeutic medicine has been developed, make people all around the world fear for infection and cause great confusion in our society and whole economy system. In an epidemic disease outbreak, it is very important for a disease control/prevention authority (such as CDC) to quickly take various quarantine measures to prevent spread/prevalence of the infection. As examples of the quarantine measures, it is well-known to timely identify an infected patient and his/her recent moving path, and also identify potentially infected persons who have been exposed to the patient's recent moving path and thus, may potentially be in a latent period of the infection, so as to timely quarantine those identified infected or potentially infected persons to stop the spread of the infection. In addition, in order to prevent spread/prevalence of the infection, it is also important to prevent the infectious viruses or germs from entering a place/facility where unspecified many people can gather, (e.g., by identifying a person with symptoms of the infectious disease, such as a high fever, and blocking accesses of such an identified person to the place/facility).

However, in a complex modern society where a large number of people visit various public places/facilities where anyone can enter, and also meet a large number of people, even though an infected patient is specified, it is very difficult to timely identify his/her recent moving path and other persons who may be contact with the infected patient on their moving paths. Since a human's memory may be insufficient, it is also insufficient to identify the moving path of the infected patient based on his/her memory. Moreover, the infected patient may not be cooperative with the disease control/prevention authority trying to identify his/her moving path. Although it may be worth considering to identify the moving path by referring to credit card usage records of the infected patient or a history of access of his/her mobile terminal to base stations as supplementary data, it is very time consuming and still insufficient in itself. Thus, it is still hard for the disease control/prevention authority to find the infected patient's recent moving path. In addition, even if the infected patient's recent moving path is specified, it requires great efforts to identify potentially infected persons who have visited any place/facility on the specified patient's moving path and thus, may potentially have contact with the infected patient. Thus, prevention of infectious diseases may not be operated quickly and promptly.

Meanwhile, in an epidemic period, as discussed above, it is important to prevent the infectious viruses or germs from entering a place/facility where unspecified many people can gather for preventing spread/prevalence of the infection. To do this, a gate manager at an entrance gate of such a place/facility may check whether each person, who wishes to enter the place/facility, has symptoms of infection (e.g., is fevered), and may restrict an access of a person with such symptoms. However, in such a case, the gate manager, who comes face to face with many people, may be exposed to potential infection so that the viruses or germs may spread among multiple access persons via the gate manager.

In relation to a recent pandemic of Coronavirus Disease 19, a national disease control/prevention authority (e.g., KCDC) have had great difficulties in finding those who are likely to have contact with an infected patient at places/facilities where a large number of unspecified persons enter and exit (for example, hospitals, public facilities, various commercial facilities such as clubs and entertainment establishments) on the moving path of the infected patient. In this regard, it is often required that each person who wishes to enter a public place/facility should record his/her name and contact information on a list. However, it makes not only people cumbersome, but also causes a high risk of leakage of each person's personal information.

### SUMMARY

Exemplary embodiments of the present disclosure provide a quarantine gate apparatus disposed at a facility access boundary of a facility that may be accessed by multiple persons, for checking a fever of each person trying to enter the facility and restricting entrance of a person having a high fever (which means that the person is likely to be infected), for providing sterilization treatments for each entering person, and for conveniently collecting and storing each identification information of each entering person in an non-contact digital manner so that the collected and stored identification information may be recollected and suitably used in a timely manner for a special public benefit such as disinfection and quarantine only by an authorized administrator (e.g. a national disinfection administrator).

According to one aspect of the present disclosure, a quarantine gate apparatus to be disposed at a boundary of a facility to be accessed by multiple users, including an inner part bordering on inside of the facility, an outer part bordering on outside of the facility; and a conditional opening gate disposed between the inner part and the outer part, wherein an entering person can reach the inside of the facility by sequentially passing through the outer part, the conditional opening gate, and the inner part is provided. The quarantine gate apparatus comprises a body temperature obtainer configured to obtain a body temperature of the entering person in a unmanned contactless manner; a body temperature analyzer configured to determine whether the body temperature is equal to or greater than a predetermined reference value; an identification information receiver configured to receive individual identification information of the entering person; an identification information storage configured to store the individual identification information of the entering person with a corresponding time information, which indicates when the identification information receiver receives the individual identification information or when the identification information storage stores the individual identification information, only for a predetermined time period, and also configured to, after the predetermined time period, permanently delete the identification information stored in the identification information storage; and a gate controller configured to control the conditional opening gate to be opened when a predetermined condition is satisfied, wherein the predetermined condition includes the body temperature analyzer determining that the body temperature of the entering person is lower than the predetermined reference value, and the identification information receiver successfully receiving the individual identification information of the entering person.

According to embodiments of the present disclosure, the quarantine gate apparatus further comprises a communicating unit configured to communicate with an external server through a network, wherein the communicating unit is configured to receive a request from the external server, to provide the individual identification information stored in the individual identification information storage, of which the corresponding time information falls within a certain time range, and wherein the communicating unit is also configured to, in response to the request from the external server, transmit the requested individual identification information to the external server.

According to embodiments of the present disclosure, the quarantine gate apparatus further comprises a sterilizer configured to spray sterilizing solution in response to sensing an object located within a predetermined proximity range around the sterilizer, wherein the predetermined condition further includes the sterilizer completing spraying the sterilizing solution for the entering person.

According to embodiments of the present disclosure, the quarantine gate apparatus further comprises a user interface configured to visually and/or audibly provide instructions on one or more actions to be taken by the entering person to pass through the quarantine gate apparatus to enter the facility.

According to embodiments of the present disclosure, the body temperature obtainer, the body temperature analyzer, the identification information receiver, the sterilizer and the user interface are disposed in the outer part.

According to embodiments of the present disclosure, the individual identification information includes at least one of information stored in a communication terminal of the entering person, information stored in an RFID chip owned by the entering person, information printed on a physical media in a visually readable form, and a biometric information of the entering person.

According to embodiments of the present disclosure, the individual identification information includes information stored in a communication terminal of the entering person in an encrypted state. The individual identification information is transmitted from the communication terminal to the identification information storage through the identification information receiver while maintaining the encrypted state, and the individual identification information is also transmitted from the identification information storage to the external server while maintaining the encrypted state.

According to embodiments of the present disclosure, the identification information receiver is configured to read the individual identification information presented on a display of the communication terminal in a form of QR code.

According to embodiments of the present disclosure, the identification information receiver is configured to wirelessly receive the individual identification information according to a short-range communication protocol from the communication terminal when the communication terminal approaches the identification information receiver within a predetermined distance.

According to embodiments of the present disclosure, the identification information receiver is further configured to receive the individual identification information in condition that the entering person explicitly indicates consent to providing of the individual identification information via the communication terminal.

According to embodiments of the present disclosure, the quarantine gate apparatus further comprises a hash generator configured to apply a hash function to a certain unit of the individual identification information stored in the identification information storage so as to obtain a hash value for the unit. In response to the request from the external server, the hash value is transmitted to the external server together with the corresponding unit of the individual identification information.

According to embodiments of the present disclosure, the quarantine gate apparatus further comprises a sterilizing blower configured to blow a sterilizing gas including a sterilized air and a sterilizing component toward the entering person; and a sterilizing nozzle configured to spray sterilizing solution to the entering person.

According to embodiments of the present disclosure, the inner part has a form of a corridor of a predetermined length, and the sterilizing blower is configured to, while the entering person is passing through the corridor of the inner part, blow the sterilizing gas to the entering person.

According to embodiments of the present disclosure, the quarantine gate apparatus further comprises a protective door disposed at an opposite end of the corridor away from the conditional opening gate. The protective door includes one of an air curtain, an access barrier to be automatically opened in response to detecting that the entering person reaches the protective door after passing the corridor, and an automatic door to be automatically opened when detecting that the sterilizing blower completes the blowing.

According to embodiments of the present disclosure, the corridor includes a moving walk that is configured to automatically move from the conditional opening gate to the protective door and to be able to simultaneously accommodate two or more entering persons who sequentially passed through the conditional opening gate.

According to embodiments of the present disclosure, the quarantine gate apparatus further comprises a plasma air sterilizer configured to absorb air from the inner part and sterilize the absorbed air by plasma generation to generate the sterilized air to be blown by the sterilizing blower.

According to embodiments of the present disclosure, the quarantine gate apparatus further comprises a sterilizing gas generator configured to generate the sterilizing gas to be blown by the sterilizing blower. The sterilizing gas generator is further configured to generate chlorine gas through electrolysis of salt water or by use of a cartridge having impregnated chlorine dioxide.

According to embodiments of the present disclosure, the quarantine gate apparatus further comprises an air cooling/heating device configured to cool or heat the sterilized air to be blown by the sterilizing blower.

According to embodiments of the present disclosure, the conditional opening gate has a form of an automatic door, and the quarantine gate apparatus further comprises a protective automatic door disposed at an opposite end of the inner part away from the conditional opening gate; a gas sensor configured to sense a concentration of the sterilizing component within an area defined between the conditional opening gate and the protective automatic door; and a blowing intensity controller configured to control the sterilizing blower such that an intensity of blowing of the sterilizing blower is decreased when the concentration of the sterilizing component sensed by the gas sensor is greater than a predetermined reference concentration and such that the intensity of blowing of the sterilizing blower is increased when the concentration of the sterilizing component sensed by the sterilizing gas sensor is less than a predetermined reference concentration.

According to embodiments of the present disclosure, the quarantine gate apparatus further comprising an air pressure controller configured to absorb air from a space between the conditional opening gate and the protective automatic door such that an air pressure in the space keeps a negative pressure which is lower than an atmospheric pressure.

According to exemplary embodiments of the present disclosure, a fever for each entering person may be checked and a person with a high fever may be restricted to access the facility. In addition, some sterilization/disinfection treatments may be provided for each entering person at an access boundary for a facility that may be accessed by multiple persons in an non-contact digital manner so that an epidemic may be efficiently prevented. According to exemplary embodiments of the present disclosure, each identification information about each entering person of the facility that may be accessed by multiple persons may be conveniently collected and stored in an non-contact digital manner so that the collected and stored identification information may be recollected and suitably used in a timely manner for a special purpose and public benefit such as disinfection and quarantine by an authorized administrator (e.g. a national disinfection administrator). Thus, the epidemic may be efficiently prevented and the socioeconomic loss of the epidemic may be minimized.

### BRIEF DESCRIPTION OF THE DRAWINGS

FIG. 1 is a schematic view illustrating a quarantine system for facilities each of which to be accessed by multiple persons, according to an exemplary embodiment of the present disclosure.

FIG. 2 is a schematic view illustrating an appearance of a gate apparatus 120 shown in FIG. 1 according to an exemplary embodiment of the present disclosure.

FIG. 3 is a schematic view illustrating a functional structure of the gate apparatus 120 shown in FIG. 1 according to an exemplary embodiment of the present disclosure.

FIG. 4 is a schematic view illustrating a functional structure of a pretreatment/gate open controller 330 of the gate apparatus 120 shown in FIG. 3 according to an exemplary embodiment of the present disclosure.

FIG. 5 is a schematic view illustrating a functional structure of a sterilizer 360 of the gate apparatus 120 shown in FIG. 3 according to an exemplary embodiment of the present disclosure.

### DETAILED DESCRIPTION OF THE INVENTIVE CONCEPT

Hereinafter, an exemplary embodiment of a present disclosure will be explained in detail with reference to the accompanying drawings.

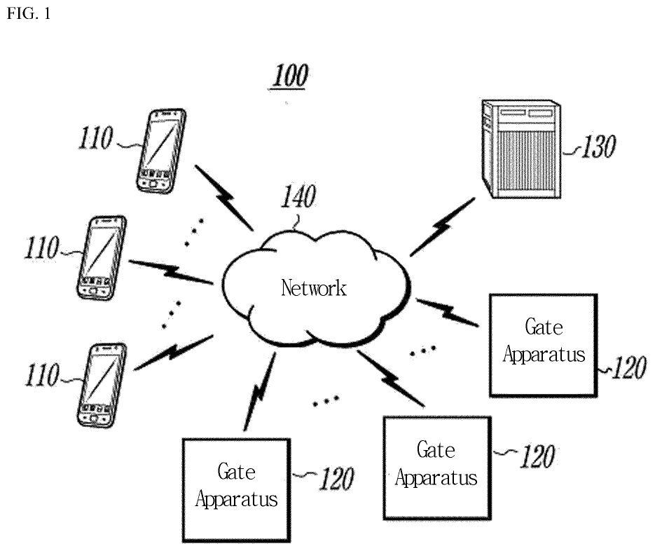

FIG. 1 is a schematic view illustrating a quarantine system related to entrance management of facilities each of which to be accessed by unspecified multiple persons, according to an exemplary embodiment of the present disclosure. Referring to FIG. 1, the quarantine system 100 includes a plurality of ID devices 110, a plurality of gate apparatus 120, a management server 130, and a network 140.

According to one embodiment of the present disclosure, the plurality of ID devices 110 may respectively store an identification information for a corresponding person, which may include at least one of a name, an address, a phone number, a date of birth, and a social security number of the person (but not limited thereto). According to one embodiment of the present disclosure, the identification information on each device 110 may be in an encrypted state according to a predetermined encryption protocol so that no body accessing the identification information cannot understand it without a special decryption key.

According to one embodiment of the present disclosure, the plurality of ID devices 110 may be a mobile communication terminal for each corresponding person. According to one embodiment of the present disclosure, the ID devices 110 may be a smartphone, a tablet, a laptop, or any of various types of communication terminals. According to one embodiment of the present disclosure, the ID devices 110 may be a mobile communication terminal including an ID management app, which may have been previously downloaded from the management server 130 (or another server which is not shown) and installed thereon. While downloading and/or installing the ID management app, the respective person may be required to input his/her identification information. While downloading and/or installing the ID management app, the respective person may be also required to undergo verification with the inputted identification information. Then, the identification information may be stored through the ID management app on the communication terminal.

According to one embodiment of the present disclosure, when a person wishes to enter a corresponding facility through the respective gate apparatus 120, he/she may operate the communication terminal (i.e., the ID device 110) to run the ID management app thereon. Alternatively, according to one embodiment of the present disclosure, the ID management app on the ID device 110 may be automatically run, without the respective person's direct intervention (such as an operation of the terminal to run the ID management app), when the person approaches the gate apparatus 120 so that the ID device 110 (i.e., the communication terminal) owned by the person detects it being located in a predetermined distance from the gate apparatus 120.

When the ID management app is run, the identification information of the entering person stored in the communication terminal may be presented on a display of the communication terminal in a visually readable form, such as a QR code. According to one embodiment of the present disclosure, the individual identification information of the entering person in the visually readable form may be read by the gate apparatus 120. According to one embodiment of the present disclosure, after the ID management app is run, the individual identification information stored in the communication terminal may be wirelessly and automatically transmitted to the gate apparatus 120 through a short-range wireless communication protocol, such as NFC. In this regard, according to one embodiment of the present disclosure, the identification information stored therein may be presented to be read by the gate apparatus 120 or wirelessly transmitted to the gate apparatus 120, only after the user of the communication terminal explicitly shows a consent to provide the identification information in the communication terminal (e.g. by clicking on a confirm consent button).

According to another embodiment of the present disclosure, the ID devices 110 may be any type of ID card or a RFID chip included therein, which stores the identification information for the corresponding person in a digital form. According to another embodiment of the present disclosure, the ID devices 110 may be a physical media, such as paper, which may include the identification information printed thereon in a visual form (e.g., a visual QR code). According to one embodiment of the present disclosure, each person may access to the management server 130 (or another server which is not shown), via any kind of communication device (not shown) and the network 140, to provide his/her identification information, including at least one of a name, an address, a phone number, a date of birth and a social security number. In response, the person may receive an encrypted identification information, for identifying the person, from the management server 130 through the communication device (not shown) and the network 140. The received identification information may be stored, via an intervention of the communication device, on the ID device 110. For example, the received identification information may be stored on the ID card or a RFID chip included therein in a digital form. Alternatively, the received identification information may be stored (or printed) on the physical media in the visually printed form.

According to one embodiment of the present disclosure, when a person tries to enter a corresponding facility through the gate apparatus 120, he/she may present the ID device 110 (e.g., the ID card including the identification information stored therein or the media having the identification information visually printed thereon) to an identification information receiver of the gate apparatus 120. Then, the identification information stored in the ID card may be wirelessly transmitted to the gate apparatus 120 through a short-range wireless communication protocol, such as NFC. Alternatively, the identification information visually printed on the physical media may be read by the identification information receiver of the gate apparatus 120.

According to one embodiment of the present disclosure, the identification information stored in the ID device 110 in the encrypted state may maintain the encrypted state while being read by or transmitted to the gate apparatus 120. After reading or receiving the identification information, the gate apparatus 120 may store and keep the identification information intact (i.e., in the encrypted state), for a predetermined period, with its respective reading/reception time (or its storing time), and after a lapse of the predetermined period, may permanently delete the identification information. While keeping the identification information, the gate apparatus 120 may transmit, if requested by a certain public purpose authority server (e.g., a CDC server for an infection prevention), the identification information with its respective reading/reception (or storing) time thereto through the network 140. According to one embodiment of the present disclosure, the certain public purpose authority server may be the management server 130, but the present disclosure is not limited thereto.

According to one embodiment of the present disclosure, the identification information may also maintain the encrypted state while being transmitted to the authority server. After the transmission to the authority server, the identification information may be decrypted with a certain decryption key by an administrator on the authority server, who is allowed to collect the identification information of each person and use the decryption key to decrypt the encrypted identification information and use the decrypted information for public purpose. Without the decryption key, nobody can read and decrypt the identification information in the encrypted state from the ID devices 110 so that the identification information stored in the communication terminal 110 may be prevented from being leaked to public.

Meanwhile, although the present disclosure mainly explains the embodiments where a gate apparatus 120 for a public facility reads/receives identification information of an unspecified person stored in his/her ID device 110, when the person tries to enter the facility through the gate apparatus 120, and maintains the read/received identification information with its reading/reception time as an access record, the present disclosure is not limited thereto. According to another embodiment of the present disclosure, the gate apparatus 120 may be an entrance apparatus for a non-public facility, i.e., a facility only for limited persons, and the gate apparatus 120 may have previously registered identification information for each of the limited persons. According to the embodiment of the present disclosure, when one of the limited person tries to enter the facility through the gate apparatus 120, the gate apparatus 120 may read or receive the identification information from the ID device 110 owned by the respective person and compare the read/received information with the respective previously-registered information to authenticate access of the corresponding person to the facility. In this embodiment, the gate apparatus 120 may operate as a security gate to allow only the limited persons, whose identification information have previously registered, to enter the facility. Then, the gate apparatus 120 may also store and maintain the read/received identification information with its reading/reception time as an access record of the person to the facility. According to one embodiment of the present disclosure, the previously registered identification information of the entering person may include bio information of the person, such as iris information, voice information and so on. The gate apparatus 120 may transmit, if requested by the certain public purpose authority server (e.g., a CDC server for an infection prevention), the access records, i.e., the read/received identification information with the corresponding reading/reception time, to the requesting server through the network 140. The certain public purpose authority server may be the management server 130, but the present disclosure is not limited thereto.

According to one embodiment of the present disclosure, as discussed above, the ID devices 110 may be a mobile communication terminal for each corresponding person. In such a case, according to one embodiment of the present disclosure, while being within the facility after passing through the gate apparatus 120, the communication terminal 110 (i.e., an ID device for an person) may operate to automatically, without any intervention of its owner, collect identification information of an approaching ID device 110 (i.e., individual identification information from another ID device 110 for another person, who is also in the facility), which approaches within a predetermined distance from the communication terminal so that the communication terminal 110 (and its owner) may closely contact the ID device 110, via a short-range wireless communication protocol. The communication terminal 110 may store and keep the received identification information for a predetermined period (e.g. a time determined based on the incubation period of an infectious disease). In addition, the communication terminal 110 may automatically delete the received identification information after the predetermined period. According to one embodiment of the present disclosure, a certain public purpose authority may collect and use, only for the public purpose, the identification information kept in the communication terminal 110 (which has been received from the approaching ID device 110 in the facility and not yet been deleted).

According to one embodiment of the present disclosure, the information received from the approaching ID device 110 may be the above-mentioned encrypted identification information of the respective person (which has been read/received by the gate apparatus 120). While being received and maintained by the communication terminal 110, the identification information from the approaching ID device 110 keeps the encrypted state.

Meanwhile, according to another embodiment of the present disclosure, the approaching ID device 110 may be also a communication terminal and then, both the communication terminals 110 approaching to each other may exchange their terminal identification information allocated to each terminal (e.g., a MAC address of each terminal), in addition to or instead of the identification information for the respective person. According to one embodiment of the present disclosure, the communication terminal 110 (which functions as an ID device) may be prevented from being turned off while the communication terminal 110 is in the facility, so that the communication terminal 110 cannot avoid exchanging the identification information with other approaching ID devices 110 within the facility.

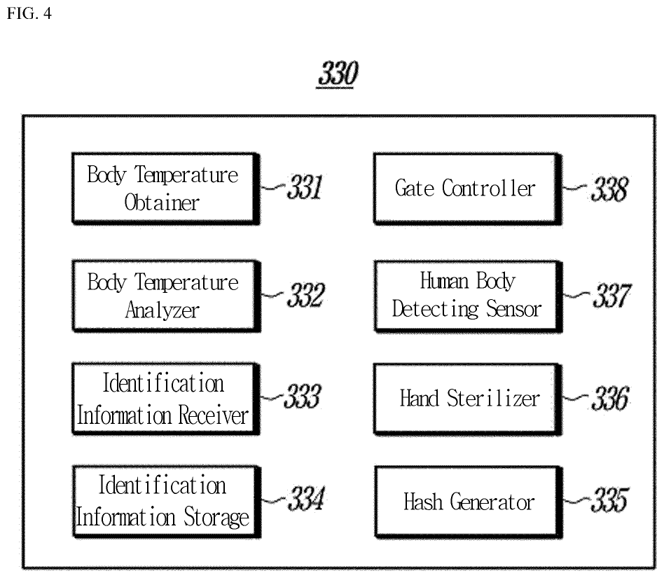

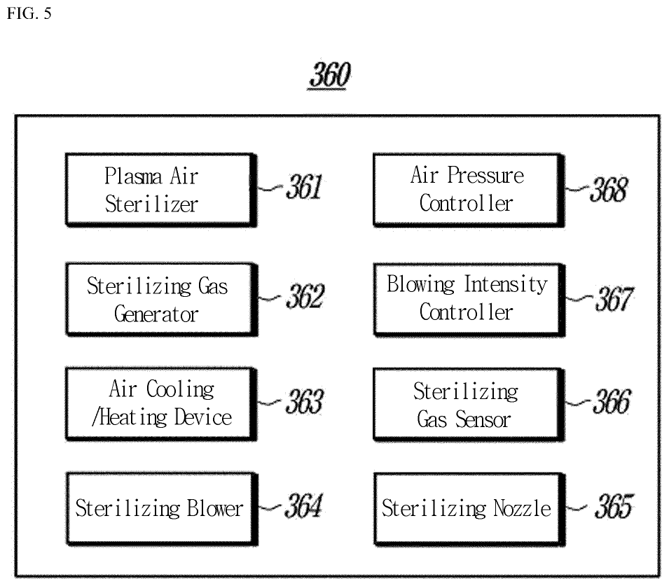

According to one embodiment of the present disclosure, each of the quarantine gate apparatus 120 may be an entrance gate apparatus disposed at a boundary between inside and outside of the facility to which multiple persons come in and out. According to one embodiment of the present disclosure, the quarantine gate apparatus 120 may include a conditional opening gate configured to be automatically opened in an non-contact manner when a predetermined condition is satisfied. The quarantine gate apparatus 120 also may include several devices disposed before or after the conditional opening gate which are configured to provide predetermined various disinfection/quarantine measures (e.g. a fever check, a hand sterilization, dry and wet sterilization measures, a collection of the identification information of the entering person and so on) in an non-contact manner for each person entering the facility, which means that the person entering the facility does not need to contact an administrator of the facility for any of the disinfection/quarantine measures. The detailed functions and the structures of the quarantine gate apparatus 120 will be further explained referring to FIGS. 4 and 5 in detail.

According to one embodiment of the present disclosure, the management server 130 may be a communicative computer device for an administrator of the quarantine system 100. According to an exemplary embodiment of the present disclosure, for example, the management server 130 may be a server device for a disease control/prevention authority (such as CDC), which manages national disinfection and quarantine management. According to an exemplary embodiment of the present disclosure, the management server 130 may request that each quarantine gate apparatus 120 transmit the identification information currently maintained therein to the management server 130 through the network 140, when such information is necessary for national public purposes, such as prevention of infectious diseases (e.g., by identifying a moving path of an infected patient and identifying potential infected persons due to contact with the infected patient) in a case of a pandemic of infectious disease such as Coronavirus Disease 19.

According to an exemplary embodiment of the present disclosure, when the epidemic of an infectious disease, if an infected patient and his/her recent moving path, including visiting facilities and corresponding visiting dates, are identified, the administrator of the management server 130 (e.g. a head of the disease control/prevention authority) may request that the quarantine gate apparatus 120 of each visiting facility of the infected patient transmit the individual identification information collected/stored therein, which have been received and stored at the patient's visiting date (or during a predetermined duration including the visiting date) through the management server 130. According to an exemplary embodiment of the present disclosure, when the management server 130 receives the individual identification information from the quarantine gate apparatus 120, the identification information may be transmitted and received in the encrypted state. According to an exemplary embodiment of the present disclosure, the management server 130 may have a decryption key. The management server 130 may decrypt the individual identification information transmitted to the management server 130 in the encrypted state using the decryption key. According to an exemplary embodiment of the present disclosure, since the management server 130 should collect and utilize personal information of each person only for a specific public purpose, such as infection prevention and disinfection, the decryption key must be secretly managed under a control of a person with a specific authority such that the personal information of persons is not leaked or abused for any other purposes.

According to an exemplary embodiment of the present disclosure, after the management server 130 construes the received identification information received from each quarantine gate apparatus 120 by decrypting them with the decryption key to identify corresponding persons, the management server 130 may provide the respective corresponding person with a notification indicating that the individual identification information thereof has been collected by the management server 130 for the specific public purpose. According to an exemplary embodiment of the present disclosure, the management server 130 may transmit the above-mentioned notification to each communication terminal owned by the respective person in various forms such as SMS, MMS and instant message through the network 140. According to an exemplary embodiment of the present disclosure, as discussed above, the communication terminal of the person may operate, while it being within a certain facility, to collect and store contact person identification information from other ID devices 110 for other persons that approach within a predetermined distance from the communication terminal within the facility. In consideration of such cases, according to an exemplary embodiment of the present disclosure, if the management server 130 identifies the communication terminal of the respective person by decrypting the received identification information from each quarantine gate apparatus 120, the management server 130 may request to the communication terminal to transmit, if any, the identification information collected from another ID devices 110 at each facility and stored in the corresponding communication terminal. The management server 130 may receive the identification information transmitted from the communication terminal 110 in response to the above-mentioned request.

According to an exemplary embodiment of the present disclosure, the network 140 may include one or more wired/wireless networks supporting one or more communication protocols. According to an exemplary embodiment of the present disclosure, the network 140 may include, for example, a short-range network such as NFC, Bluetooth, Wi-Fi, TCP/IP networks such as LAN and WAN and various mobile networks such as CDMA, OFDM, and

GSM. However, the present disclosure may not be limited to the above-mentioned networks.

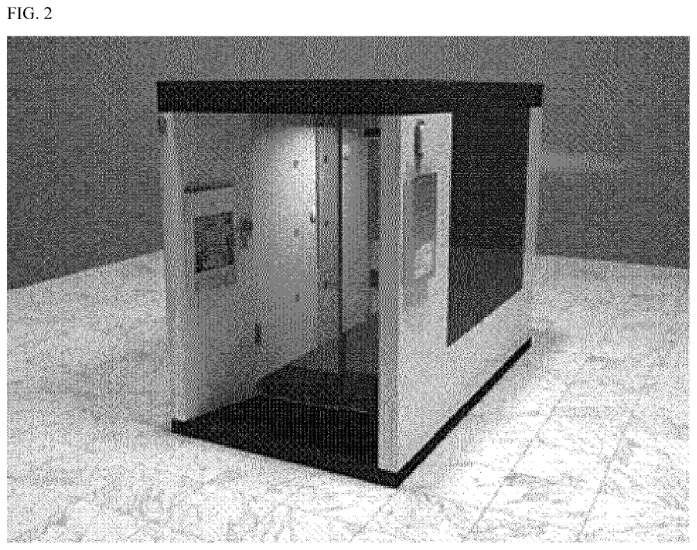

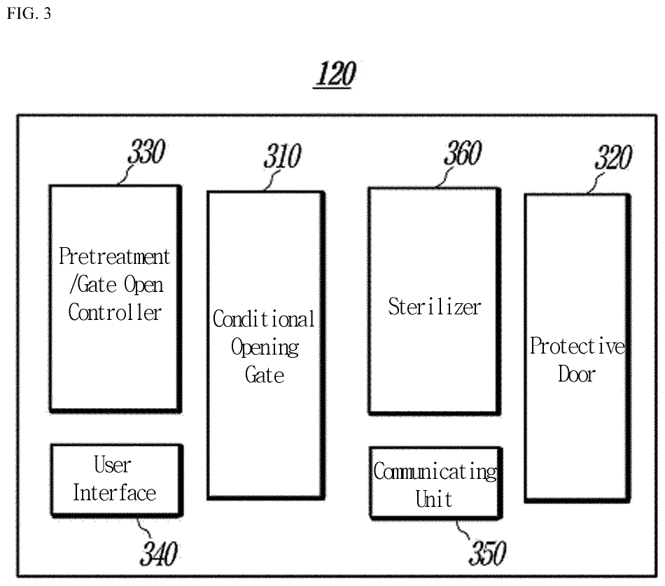

FIG. 2 is a schematic view illustrating an appearance of a quarantine gate apparatus 120 shown in FIG. 1 according to an exemplary embodiment of the present disclosure. FIG. 3 is a schematic view illustrating a functional structure of the quarantine gate apparatus 120 shown in FIG. 1 according to an exemplary embodiment of the present disclosure. Referring to FIG. 3, the quarantine gate apparatus 120 includes the conditional opening gate 310, a protective door 320, a pretreatment/gate open controller 330, a user interface 340, a communicating unit 350, and a sterilizer 360.

According to an exemplary embodiment of the present disclosure, the conditional opening gate 310 may be a gate (e.g. an automatic door or a blocking gate) which is automatically opens and closes without an operation of the person or any contact to the conditional opening gate 310, after the person accesses the quarantine gate apparatus 120 and if a predetermined condition is satisfied. According to an exemplary embodiment of the present disclosure, the quarantine gate apparatus 120 may include an outer part with respect to the conditional opening gate and an inner part with respect to the conditional opening gate. The inner part is disposed adjacent to the inside portion of the facility. According to an exemplary embodiment of the present disclosure, the quarantine gate apparatus 120 may include the protective door 320 disposed after the conditional opening gate 310 in a direction from the outside of the facility to the inside of the facility (the protective door 320 is disposed at another end portion opposite to the conditional opening gate 310 and disposed in the inside portion of the facility.) According to an exemplary embodiment of the present disclosure, the protective door 320 is an air curtain for the person to pass through, after passing through the conditional opening gate 310 and the inner part sequentially. Alternatively, the protective door 320 may be an access breaker which can be automatically opened and closed when the person reaches close to the protective door after passing through the inner part. According to an exemplary embodiment of the present disclosure, the protective door 320 may be an automatic door which is automatically opened and closed after a sterilizing process with the sterilizer 360 to the person who passed through the conditional opening gate 310 is completed.

According to an exemplary embodiment of the present disclosure, when a limited space is formed between the conditional opening gate and the protective door (e.g., when both the conditional opening gate 310 and the protective door 320 have a form of the automatic door and a closed area is generated by closing both the conditional opening gate 310 and the protective door 320), the quarantine gate apparatus 120 may include a manual opening and closing device (not shown) disposed at the conditional opening gate 310 so that a person located in the limited space may manually open the conditional opening gate 310 in an emergency, but the present disclosure may not be limited to thereto. In addition, according to an exemplary embodiment of the present disclosure, the quarantine gate apparatus 120 may include an emergency bell (not shown) disposed in the space between the conditional opening gate 310 and the protective door 320 which allows the person located in the space to provide an alarm to an emergency manager in an emergency, but the present disclosure may not be limited thereto.

In addition, according to an exemplary embodiment of the present disclosure, the space (the inner part) between the conditional opening gate 310 and the protective door 320 in the quarantine gate apparatus 120 may form a passage or a corridor having a predetermined length (e.g., about 5 meters length). According to an exemplary embodiment of the present disclosure, when the person passes through the conditional opening gate 310 of the quarantine gate apparatus 120 and moves toward the protective door 320, the sterilization process may be applied to the moving person by the sterilizer 360. According to an exemplary embodiment of the present disclosure, the quarantine gate apparatus 120 may include a moving walk (not shown) automatically moving from the conditional opening gate 310 to the protective door 320 in the inner part. According to an exemplary embodiment of the present disclosure, the conditional opening gate 310 is opened to allow the person to enter the inner part of the gate apparatus 120 when a predetermined condition is satisfied. Then, after a predetermined time or more after opening (and closing) for the (previous) person goes by, the conditional opening gate 310 may be opened again to allow a next person to enter the inner part of the gate apparatus 120 when a next condition is satisfied. Thus, according to an exemplary embodiment of the present disclosure, two or more persons may be present simultaneously inside the gate apparatus 120 (e.g., in the inner part having a form of the passage), but these persons may be guided to maintain a predetermined safety distance (e.g. 2 meters) within the inner part. In the case where the movement of the persons in the inner part is performed through the moving walk, maintaining a gap between the persons may be naturally induced even if a plurality of persons are simultaneously located in the inner part.

According to an exemplary embodiment of the present disclosure, the pretreatment/gate open controller 330 may control an opening of the conditional opening gate 310. According to an exemplary embodiment of the present disclosure, the pretreatment/gate open controller 330 may provide some pretreatments in an non-contact manner for the person who approaches the quarantine gate apparatus 120 to enter the facility. According to an exemplary embodiment of the present disclosure, the pretreatment/gate open controller 330 may measure or obtain a body temperature of the person who approaches the outer part of the quarantine gate apparatus 120 to enter the facility, in a non-face-to-face and non-contact manner, and determine whether the measured body temperature is within a normal range.

According to an exemplary embodiment of the present disclosure, the pretreatment/gate open controller 330 may control a human body detectable disinfectant spraying device (e.g. human body detectable hand sanitizer dispenser) disposed at the outer part of the quarantine gate apparatus 120 (not shown) to spray a disinfectant to the person. According to an exemplary embodiment of the present disclosure, the pretreatment/gate open controller 330 may read or receive the identification information from the entering person in an non-contact manner and may store the identification information. According to an exemplary embodiment of the present disclosure, when all the above-mentioned pretreatments, such as the body temperature measurement and normal range check, spraying the sanitizer and the normal reception of the identification information of the person, are completed and the pretreatment/gate open controller 330 detects the person existing within a predetermined distance from the pretreatment/gate open controller 330, the pretreatment/gate open controller 330 may determine that the predetermined condition is satisfied and may control to open the conditional opening gate 310. According to an exemplary embodiment of the present disclosure, the conditional opening gate 310 may be closed after a preset time, which is set to be enough for the person to pass through, lapses.

According to an exemplary embodiment of the present disclosure, the user interface 340 may include an output interface device disposed at the outer part of the quarantine gate apparatus 120. According to an exemplary embodiment of the present disclosure, the user interface 340 may provide guidance or information to guide the person to take sequential procedures to enter the facility through the quarantine gate apparatus 120 in a visual and/or auditory method (e.g. a voice, an image, a video and a text). According to an exemplary embodiment of the present disclosure, for example, the user interface 340 may include a speaker and a display device.

According to an exemplary embodiment of the present disclosure, the communicating unit 350 may support the quarantine gate apparatus 120 to perform wired or wireless communication with the management server 130 according to one or more communication protocols. According to an exemplary embodiment of the present disclosure, the quarantine gate apparatus 120 may access the management server 130 through the communicating unit 350 according to a predetermined communication protocol (e.g. TCP/IP protocol, etc.), and may transmit and receive the information to/from the management server 130. According to an exemplary embodiment of the present disclosure, a facility manager (e.g. a business owner, etc.) may turn on the quarantine gate apparatus 120 when initiating the operation of the facility and thus, a signal indicating the initiation of the operation of the facility (e.g. a business place) on the date may be transmitted to the management server 130. According to an exemplary embodiment of the present disclosure, the quarantine gate apparatus 120 may receive a request for access records collected and stored in the quarantine gate apparatus 120 (i.e., the identification information of the entering persons) from the management server 130 through the communicating unit 350. The quarantine gate apparatus 120 may transmit the identification information of the persons to the management server 130 in response to the received request. According to an exemplary embodiment of the present disclosure, when the quarantine gate apparatus 120 provides the access records, i.e., the identification information of the persons stored in the quarantine gate apparatus 120 to the management server 130 through the communicating unit 350, the quarantine gate apparatus 120 may also transmit hash values stored in association with the corresponding individual identification information to the management server 130.

According to an exemplary embodiment of the present disclosure, the sterilizer 360 may provide dry and/or wet sterilization treatments to the person who is in the inner part between the conditional opening gate 310 and the protective door 320 after passing through the conditional opening gate 310. According to an exemplary embodiment of the present disclosure, the sterilizer 360 may provide a dry sterilization to the person by blowing a sterilizing gas, which may be a mixture of sterilized fresh air and sterilizing component (e.g., chlorine) to the inner part area where the person is existing for a predetermined time. According to an exemplary embodiment of the present disclosure, the sterilizer 360 may provide a wet sterilization by spraying a small amount of a sterilizing solution including various sterilizing components, such as underwater plasma OH-radical and sodium hypochlorite, to the person.

FIG. 4 is a schematic view illustrating a functional structure of the pretreatment/gate open controller 330 of the quarantine gate apparatus 120 shown in FIG. 3 according to an exemplary embodiment of the present disclosure. As shown in FIG. 4, the pretreatment/gate open controller 330 includes a body temperature obtainer 331, a body temperature analyzer 332, an identification information receiver 333, an identification information storage 334, a hash generator 335, a hand sterilizer 336, a human body detecting sensor 337 and a gate controller 338.

According to an exemplary embodiment of the present disclosure, the body temperature obtainer 331 may be a device that measures the body temperature in a non-face-to-face (non-contact) manner for an person approaching the outer part of the quarantine gate apparatus 120. According to an exemplary embodiment of the present disclosure, for example, the body temperature obtainer 331 may measure the person's body temperature using an infrared thermal imaging camera installed in the quarantine gate apparatus 120. Alternatively, according to an exemplary embodiment of the present disclosure, the body temperature obtainer 331 may be configured to receive from a external body temperature measuring device (e.g. a separate mobile or fixed thermal camera, etc.) disposed on a outside moving path of the person, instead of measuring the body temperature of the person.

According to an exemplary embodiment of the present disclosure, the temperature analyzer 332 may determine whether the body temperature obtained by the body temperature obtainer 331 is normal (i.e., being within a preset safe range, without a high fever causing suspect of potential infection). According to an exemplary embodiment of the present disclosure, for example, the body temperature analyzer 332 may compare the obtained body temperature of the person with a predetermined reference value, and may determine the obtained body temperature as being normal when the obtained body temperature is less than the reference value. According to an exemplary embodiment of the present disclosure, the body temperature analyzer 332 may consider air temperature to set the above-mentioned predetermined reference value. For example, when the air temperature is equal to or greater than a predetermined value (for example, equal to or greater than 0° C.), the predetermined reference value may be set to any value (for example, 37.5° C. above an ideal body temperature). In contrast, when the air temperature is less than the predetermined value (for example, under 0° C.) (then the body temperature may be measured relatively low), the predetermined reference value may be adjusted to another value (for example, a value determined based on an average body temperature of a plurality of persons who entered the facility before the corresponding person on the day or on the day before). According to an exemplary embodiment of the present disclosure, when the body temperature of the person is determined as out of the normal range (e.g. equal to or greater than the predetermined reference value), a warning alarm may be provided through the user interface 340. According to an exemplary embodiment of the present disclosure, when the body temperature of the person is determined as out of the normal range (e.g. equal to or greater than the predetermined reference value), an alarm may be provided to an administrator, but the present disclosure may not be limited thereto.

According to an exemplary embodiment of the present disclosure, the identification information receiver 333 may be a device that can read or wirelessly receive the identification information from the ID device 110 of the person who is entering the facility. According to an exemplary embodiment of the present disclosure, the identification information stored in the ID device 110 may be presented in a form of a QR code or the like on a display of the ID device 110 (if the ID device 110 is a device having the display, like a communication terminal) and the identification information receiver 333 may be capable of reading the presented QR code or the like. According to an exemplary embodiment of the present disclosure, the identification information receiver 333 may be a wireless information reader that can receive the identification information of the person, according to a short-range wireless communication (e.g. NFC communication, etc.) protocol, transmitted from the ID device 110 which is located within a predetermined distance near the identification information receiver 333.

According to an exemplary embodiment of the present disclosure, the identification information storage 334 may be a recording device which stores the individual identification information of the person read and received by the identification information receiver 333.

According to an exemplary embodiment of the present disclosure, as explained above, the identification information of the person may be stored in the identification information storage 334 in an encrypted state. According to an exemplary embodiment of the present disclosure, the identification information of the person stored in the identification information storage 334 may be automatically deleted after a predetermined time period from the time of the storage of the information (for example, the predetermined time period that is deemed necessary to identify a moving path of a infected patient considering the incubation period of the infectious disease).

According to an exemplary embodiment of the present disclosure, the hash generator 335 may apply a predetermined hash function to each storage unit of the identification information stored in the identification information storage 334. According to an exemplary embodiment of the present disclosure, the identification information of the entering persons may be stored in the identification information storage 334 by a certain unit (for example, the identification information collected on a certain date (or any other period) may by grouped and stored as a unit) and the hash generator 335 may apply the hash function to each unit of the stored identification information (for example, the identification information collected on a certain date may be grouped and stored as a unit, when the operation of the corresponding facility is terminated and the quarantine gate apparatus 120 is turned off, and then, the hash generator 335 may apply the hash function to the unit of the identification information collected during that day). According to another exemplary embodiment of the present disclosure, the hash generator 335 may apply the hash function to each unit of the identification information and may store the result value in association with the storage of the identification information.

According to an exemplary embodiment of the present disclosure, the hand sterilizer 336 may be a body detecting type hand sanitizer spraying device that detects that the person's body is in a predetermined position. According to an exemplary embodiment of the present disclosure, the hand sterilizer 336 may spray a predetermined amount of hand sanitizer in a non-contact automatic manner when the person's hand located under a dispensing spout of the hand sterilizer 336 is detected. According to an exemplary embodiment of the present disclosure, the hand sterilizer 336 may provide alcohol, chlorine-based sterilizing solution and hydrogen-based sterilizing solution. However, the present disclosure may not be limited to the above-mentioned hand sanitizers.

According to an exemplary embodiment of the present disclosure, the human body detecting sensor 337 may be any type of human body detecting sensor disposed near the conditional opening gate 310. According to an exemplary embodiment of the present disclosure, the human body detecting sensor 337 may be configured to detect whether a human body is located on a trajectory where the conditional opening gate 310 is opened and closed. When the gate controller 338 receives a signal that the human body detecting sensor 337 sensed the human body on the trajectory of the conditional opening gate 310, the gate controller 338 may control the conditional opening gate 310 to maintain an opened state for a predetermined time to avoid a crash accident. According to an exemplary embodiment of the present disclosure, when the gate controller 338 receives a determination result from the body temperature analyzer 332 that the body temperature of the person is within a normal range, receives notification from the identification information receiver 333 that the identification information of the person is normally received (and stored), and receives notification by the hand sterilizer 336 that spraying of the hand sanitizer is completed, the gate controller 338 may determine that the condition is satisfied and may control the conditional gate controller 338 to be opened.

FIG. 5 is a schematic view illustrating a functional structure of the sterilizer 360 of the quarantine gate apparatus 120 shown in FIG. 3 according to an exemplary embodiment of the present disclosure. As shown in FIG. 5, the sterilizer 360 includes a plasma air sterilizer 361, a sterilizing gas generator 362, an air cooling/heating device 363, a sterilizing blower 364, a sterilizing nozzle 365, a sterilizing gas sensor 366, a blowing intensity controller 367 and an air pressure controller 368.

According to an exemplary embodiment of the present disclosure, the plasma air sterilizer 361 may absorb air from outside, and sterilize viruses and germs by continuously heating the absorbed air to generate plasma. According to an exemplary embodiment of the present disclosure, the plasma air sterilizer 361 may include a HEPA filter or the like for collecting dust of air absorbed from the outside, and the present disclosure may not be limited thereto.

According to an exemplary embodiment of the present disclosure, the sterilizing gas generator 362 may generate a sterilizing gas in various ways. According to an exemplary embodiment of the present disclosure, the sterilizing gas generator 362 may generate chlorine gas via a salt-water electrolysis method. According to an exemplary embodiment of the present disclosure, the sterilizing gas generator 362 may generate chlorine gas by providing a predetermined temperature condition and a predetermined humidity condition (e.g. 40% humidity, etc.) to a disinfectant cartridge having chlorine dioxide impregnated in a powder form and chlorine dioxide gel. According to another exemplary embodiment of the present disclosure, the sterilizing gas generator 362 may generate a sterilizing gas including chlorine gas in various generating methods using any of various raw materials.

According to an exemplary embodiment of the present disclosure, the air cooling/heating device 363 may be an air cooling/heating device that cools or heats the temperature of the flowing air to a comfortable temperature. As not shown in FIG. 5 in detail, for example, the air cooling/heating device 363 may include a device which operates an air cooling process through a series of processes of compression, condensation, expansion, and evaporation of refrigerant. As not shown in FIG. 5 in detail, for example, the air cooling/heating device 363 may include a heater device which heats the air to an appropriate temperature in any manner.

According to an exemplary embodiment of the present disclosure, the sterilizing blower 364 may include a blower device that blows air into the inner part between the conditional opening gate 310 and the protective door 320. According to an exemplary embodiment of the present disclosure, the air sterilized by the plasma air sterilizer 361 is mixed with the sterilizing component generated by the sterilizing gas generator 362, and the mixture of the sterilized air and the sterilizing component may be cooled by the air cooling/heating device 363 on an air flow path (not shown) inside a wall of the quarantine gate apparatus 120 (not exposed to the user), and this sterilized and cooled air (and sterilizing component) may be blown into the inner part between the conditional opening gate 310 and the protective door 320 by the sterilizing blower 364. As a result, dry sterilization of the person standing in the inner part between the conditional opening gate 310 and the protective door 320 may be performed. In the drawings and description, the air cooled/heated by the air cooling/heating device 363 is blown by the sterilizing blower 364, but the present disclosure is not limited thereto. In addition, in the drawings and description, the flow in the air flow path (not shown) inside the wall of the quarantine gate apparatus 120 is in the order of air sterilization by plasma, generation and mixing of sterilizing gas, and cooling of air, but the present disclosure is not limited thereto. Alternatively, the order of the air flow may be changed.

According to an exemplary embodiment of the present disclosure, the sterilizing nozzle 365 may include one or more nozzles spraying the sterilizing solution toward the inner part between the conditional opening gate 310 and the protective door 320. According to an exemplary embodiment of the present disclosure, the sterilizing nozzle 365 may include a mist spray nozzle spraying hydrogen-based disinfecting solution (disinfecting solution containing hydrogen peroxide generated by underwater plasma, etc.), alcohol and chlorine-based disinfecting solution (disinfecting solution containing components such as sodium chlorite, hypochlorous acid and chlorine dioxide, etc.). The sterilizing solution sprayed by the sterilizing nozzle 365 may be sprayed in a mist form or an aerosol form, and the present disclosure is not limited thereto.

As explained above, the inner part between the conditional opening gate 310 and the protective door 320 may have a long passage shape (in which a plurality of persons passing between the conditional opening gate 310 and the protective door 320 may exist simultaneously in the inner part). In this case, blowing of the sterilized air and the sterilizing gas by the sterilizing blower 364 and spraying of the sterilizing solution by the sterilizing nozzle 365 may be performed in a predetermined sequence for the person passing through the inner part having the long passage shape.

According to an exemplary embodiment of the present disclosure, the sterilizing gas sensor 366 may be a sensor configured to sense a concentration of the sterilizing component at the inner part between the conditional opening gate 310 and the protective door 320 where the sterilized air and the sterilizing component are provide by the sterilizing blower 364. According to an exemplary embodiment of the present disclosure, when the concentration of the sterilizing component sensed by the sterilizing gas sensor 366 is equal to or greater than a predetermined reference concentration (for example, the reference concentration is considered to secure safety for the human body), the blowing intensity controller 367 may control the sterilizing blower 364 to decrease the intensity of the blowing of the sterilizing blower 364. According to an exemplary embodiment of the present disclosure, when the concentration of the sterilizing component sensed by the sterilizing gas sensor 366 is less than a predetermined reference concentration (for example, the reference concentration recognized as having an effective sterilizing effect), the blowing intensity controller 367 may control the sterilizing blower 364 to increase the intensity of the blowing of the sterilizing blower 364.

According to an exemplary embodiment of the present disclosure, the air pressure controller 368 is configured to absorb air at the inner part between the conditional opening gate and the protective door such that an air pressure in the inner part maintains a negative pressure which is lower than an atmospheric pressure. As described above, by forming the space between the conditional opening gate 310 and the protective door 320 as a negative pressure space, pathogenic substances such as viruses or germ, which may have existed in the corresponding area, may be prevented from penetrating inside the facility passing through the protective door 320.

The quarantine gate apparatus 120 according to embodiments of the present disclosure may be provided at entrance boundary between inside and outside of various indoor/outdoor facilities which can be used by a large number of people, such as various commercial facilities, performance halls, various buildings, schools, public institutions, tourist attractions, parks, and various sports facilities. The quarantine gate apparatus 120 according to embodiments of the present disclosure may provide a disinfection and quarantine procedure for users entering such facilities in a non-contact manner (without requiring an on-site administrator). For example, when the quarantine gate apparatus 120 according to an exemplary embodiment of the present disclosure is used, a person who is at high risk of infection, such as a person with high fever, may be picked up and blocked from entering the facility, and various types of sterilization and disinfection, such as hand disinfection, dry/wet sterilization, may be essentially applied to persons of the normal-range body temperature before entry. The above procedures may be done in a non-face-to-face manner and in a non-contact manner without the on-site administrator. In addition, the quarantine gate apparatus 120 according to an exemplary embodiment of the present disclosure may control only users who provide their identification information to enter the facility, so that identification information of all users who have access to the facility can be easily collected without the on-site administrator. The quarantine gate apparatus 120 according to an exemplary embodiment of the present disclosure ensures that the collected personal/individual identification information is not abused and only used for a limited public purpose by a person with a certain authority for the purpose of specific public purposes, so that appropriate disinfection and quarantine measures may be timely achieved according to both personal and public interests.

As will be appreciated by those skilled in the art, the present invention is not limited to the examples described in the present disclosure, but can be variously modified, reconstructed, and replaced without departing from the scope of the present disclosure. It should be understood that the various techniques described herein can be implemented by hardware or software, or a combination of hardware and software.

What is claimed is:

1. A quarantine gate apparatus to be disposed at a boundary of a facility to be accessed by multiple users, including an inner part bordering on inside of the facility, an outer part bordering on outside of the facility; and a conditional opening gate disposed between the inner part and the outer part, wherein an entering person can reach the inside of the facility by sequentially passing through the outer part, the conditional opening gate, and the inner part;
the quarantine gate apparatus comprising,
a body temperature obtainer configured to obtain a body temperature of the entering person in a unmanned contactless manner;
a body temperature analyzer configured to determine whether the body temperature is equal to or greater than a predetermined reference value;
an identification information receiver configured to receive individual identification information of the entering person;
an identification information storage configured to store the individual identification information of the entering person with a corresponding time information, which indicates when the identification information receiver receives the individual identification information or when the identification information storage stores the individual identification information, only for a predetermined time period, and also configured to, after the predetermined time period, permanently delete the identification information stored in the identification information storage; and
a gate controller configured to control the conditional opening gate to be opened when a predetermined condition is satisfied, wherein the predetermined condition includes the body temperature analyzer determining that the body temperature of the entering person is lower than the predetermined reference value, and the identification information receiver successfully receiving the individual identification information of the entering person.

2. The quarantine gate apparatus of claim 1, further comprising a communicating unit configured to communicate with an external server through a network,
wherein the communicating unit is configured to receive a request from the external server, to provide the individual identification information stored in the individual identification information storage, of which the corresponding time information falls within a certain time range, and wherein the communicating unit is also configured to, in response to the request from the external server, transmit the requested individual identification information to the external server.

3. The quarantine gate apparatus of claim 1, further comprising a sterilizer configured to spray sterilizing solution in response to sensing an object located within a predetermined proximity range around the sterilizer,
wherein the predetermined condition further includes the sterilizer completing spraying the sterilizing solution for the entering person.

4. The quarantine gate apparatus of claim 1, further comprising a user interface configured to visually and/or audibly provide instructions on one or more actions to be taken by the entering person to pass through the quarantine gate apparatus to enter the facility.

5. The quarantine gate apparatus of claim 4, wherein the body temperature obtainer, the body temperature analyzer, the identification information receiver, the sterilizer and the user interface are disposed in the outer part.

6. The quarantine gate apparatus of claim 1, wherein the individual identification information includes at least one of information stored in a communication terminal of the entering person, information stored in an RFID chip owned by the entering person, information printed on a physical media in a visually readable form, and a biometric information of the entering person.

7. The quarantine gate apparatus of claim 2, wherein the individual identification information includes information stored in a communication terminal of the entering person in an encrypted state,
wherein the individual identification information is transmitted from the communication terminal to the identification information storage through the identification information receiver while maintaining the encrypted state, and
wherein the individual identification information is also transmitted from the identification information storage to the external server while maintaining the encrypted state.

8. The quarantine gate apparatus of claim 6, wherein the identification information receiver is configured to read the individual identification information presented on a display of the communication terminal in a form of QR code.

9. The quarantine gate apparatus of claim 6, wherein the identification information receiver is configured to wirelessly receive the individual identification information according to a short-range communication protocol from the communication terminal when the communication terminal approaches the identification information receiver within a predetermined distance.

10. The quarantine gate apparatus of claim 6, wherein the identification information receiver is further configured to receive the individual identification information in condition that the entering person explicitly indicates consent to providing of the individual identification information via the communication terminal.

11. The quarantine gate apparatus of claim 2, further comprising a hash generator configured to apply a hash function to a certain unit of the individual identification information stored in the identification information storage so as to obtain a hash value for the unit, and wherein, in response to the request from the external server, the hash value is transmitted to the external server together with the corresponding unit of the individual identification information.

12. The quarantine gate apparatus of claim 1, further comprising:
a sterilizing blower configured to blow a sterilizing gas including a sterilized air and a sterilizing component toward the entering person; and
a sterilizing nozzle configured to spray sterilizing solution to the entering person.

13. The quarantine gate apparatus of claim 12, wherein the inner part has a form of a corridor of a predetermined length, and
wherein the sterilizing blower is configured to, while the entering person is passing through the corridor of the inner part, blow the sterilizing gas to the entering person.

14. The quarantine gate apparatus of claim 13, further comprising a protective door disposed at an opposite end of the corridor away from the conditional opening gate,
wherein the protective door includes one of an air curtain, an access barrier to be automatically opened in response to detecting that the entering person reaches the protective door after passing the corridor, and an automatic door to be automatically opened when detecting that the sterilizing blower completes the blowing.

15. The quarantine gate apparatus of claim 14, wherein the corridor includes a moving walk that is configured to automatically move from the conditional opening gate to the protective door and to be able to simultaneously accommodate two or more entering persons who sequentially passed through the conditional opening gate.

16. The quarantine gate apparatus of claim 12, further comprising a plasma air sterilizer configured to absorb air from the inner part and sterilize the absorbed air by plasma generation to generate the sterilized air to be blown by the sterilizing blower.

17. The quarantine gate apparatus of claim 12, further comprising a sterilizing gas generator configured to generate the sterilizing gas to be blown by the sterilizing blower,
wherein the sterilizing gas generator is further configured to generate chlorine gas through electrolysis of salt water or by use of a cartridge having impregnated chlorine dioxide.

18. The quarantine gate apparatus of claim 12, further comprising an air cooling/heating device configured to cool or heat the sterilized air to be blown by the sterilizing blower.

19. The quarantine gate apparatus of claim 12, wherein the conditional opening gate has a form of an automatic door, and the quarantine gate apparatus further comprising:
a protective automatic door disposed at an opposite end of the inner part away from the conditional opening gate;
a gas sensor configured to sense a concentration of the sterilizing component within an area defined between the conditional opening gate and the protective automatic door; and
a blowing intensity controller configured to control the sterilizing blower such that an intensity of blowing of the sterilizing blower is decreased when the concentration of the sterilizing component sensed by the gas sensor is greater than a predetermined reference concentration and such that the intensity of blowing of the sterilizing blower is increased when the concentration of the sterilizing component sensed by the gas sensor is less than a predetermined reference concentration.

20. The quarantine gate apparatus of claim 19, further comprising an air pressure controller configured to absorb air from a space between the conditional opening gate and the protective automatic door such that an air pressure in the space keeps a negative pressure which is lower than an atmospheric pressure.
